(19) United States
(12) Patent Application Publication
(10) Pub. No.: US 2018/0135110 A1
(43) Pub. Date: May 17, 2018

(54) DEVICES AND METHODS TO DETECT BIOMARKERS USING OLIGONUCLEOTIDES

(71) Applicant: POC MEDICAL SYSTEMS INC, Livermore, CA (US)

(72) Inventors: Sanjeev Saxena, Livermore, CA (US); Louis Eugene Burton, San Mateo, CA (US); Andrea Cuppoletti, Livermore, CA (US)

(73) Assignee: POC MEDICAL SYSTEMS INC, Livermore, CA (US)

(21) Appl. No.: 15/832,635

(22) Filed: Dec. 5, 2017

Related U.S. Application Data
(63) Continuation of application No. PCT/US2016/038668, filed on Jun. 22, 2016.

(51) Int. Cl. C12Q 1/6811; C12Q 1/6806; C12Q 1/686

(52) U.S. Cl. CPC C12Q 1/6811; C12Q 1/6806; C12Q 2600/158; C12N 2310/16; C12N 2310/3519; C12Q 1/686

(57) ABSTRACT

Devices for detecting targets in a sample and methods for detecting such targets are discussed. The targets may be biomarkers associated with a disease or other health condition. The devices may contain a disc including a plurality of microfluidic channels each extending in a radial direction of the disc, the microfluidic channels containing a plurality of capture molecules specific to at least one target. The capture molecules may include an aptamer and/or an oligonucleotide capable of hybridizing to the target. The methods may include introducing a fluid sample into one or more microfluidic channels of a disc, rotating the disc, such that the fluid sample flows radially outward through the microfluidic channel(s) to combine with capture molecules in the disc, and detecting a signal from the disc indicative of a presence of the target.

### CROSS REFERENCE TO RELATED APPLICATIONS

This application claims the benefit of priority to and is a continuation of PCT/US2016/038668 filed on 22 Jun. 2016 which is incorporated by reference herein in its entirety.

### TECHNICAL FIELD

The present disclosure generally relates to the detection of biomarkers associated with a health condition, e.g., to assist in medical screening and/or diagnosis.

### BACKGROUND

Biomarkers and other analytes can provide useful medical and/or diagnostic information. Yet, diseases and other health conditions can involve numerous biochemical species and reactions. For example, breast cancer is a complex disease which can have multiple pathways to generate the same stage of disease with similar symptoms for the patient. While researchers have sought new biomarkers, the ability to screen for various diseases remains limited. Research over the past decade has focused on discovering new biomarkers to provide accurate diagnosis of disease, guide therapeutic decision making, and predict future patterns of disease. Yet some diseases like breast cancer may be not a single disease, but a genetically heterogeneous set of diseases. For such conditions, it may be difficult or not possible to diagnose with a single biomarker. Detection and quantification of specific analytes can present additional hurdles, especially given a growing need for prompt diagnostic information.

### SUMMARY

The present disclosure includes devices comprising a disc including a plurality of microfluidic channels extending in a radial direction of the disc, each microfluidic channel comprising a plurality of capture molecules specific to at least one target chosen from an oligonucleotide, a protein, or a small molecule, wherein each capture molecule comprises an oligonucleotide and each capture molecule is attached to a substrate. In some examples, the plurality of capture molecules may include at least one aptamer, an oligonucleotide comprising a sequence at least partially complementary or fully complementary to a sequence of the target or targets, and/or at least one chimeric molecule comprising an oligonucleotide. In some aspects, each oligonucleotide of the plurality of capture molecules may comprise a sequence at least partially complementary or fully complementary to a sequence of the at least one target.

The plurality of capture molecules may comprise natural nucleotides, synthetic nucleotides, or a combination thereof. Further, the oligonucleotides of the capture molecules may have a length ranging from 5 to 10,000 nucleotides, such as from 20 to 5,000 nucleotides, or from 100 to 1,000 nucleotides. In some examples, the plurality of capture molecules may comprise DNA and/or RNA, or a fragment of DNA and/or RNA.

According to some aspects of the present disclosure, the substrate may comprise a microarray or a plurality of microbeads. For example, the substrate may comprise a microarray that includes one or more types of capture molecules arranged into discrete groupings or distributed across the surface of the microarray. The microarray may include capture molecules specific to at least one target, at least two different targets, or at least three different targets, which may be arranged into discrete areas or features or distributed across the surface of the microarray. In some examples, the plurality of capture molecules may include a first plurality of capture molecules specific to a first target attached to a first area of the microarray and a second plurality of capture molecules specific to a second target attached to a second area of the microarray. For example, the first area may include two or more discrete features on the microarray defined by a grouping of the first plurality of capture molecules. Similarly, the second area may include two or more discrete features on the microarray defined by a grouping of the second plurality of capture molecules. When microbeads are used as substrates, the microbeads may have an average diameter ranging from about 10 nm to about 100 μm, such as an average diameter ranging from 100 nm to 10 μm.

The target or targets of a sample to be detected with the device may comprise biomarkers associated with a disease or other health condition. Thus, for example, the plurality of capture molecules may include capture molecules specific to one or more biomarkers indicative of a disease. According to some aspects of the present disclosure, the plurality of capture molecules may include capture molecules specific to biomarkers indicative of cancer, a cardiac disease, a respiratory disease, a neurological disease, an infectious disease, or antibiotic resistant genes. With respect to infectious diseases, for example, the plurality of capture molecules may include capture molecules specific to pathogens associated with an infectious disease.

Further, in some examples, the disc may contain capture molecules specific to different diseases or health conditions, e.g., a first microfluidic channel including a plurality of first capture molecules specific to a first target and a second microfluidic channel including a plurality of second capture molecules specific to a second target different from the first target. The first and second targets may be biomarkers of the same or different diseases or other health condition.

The microfluidic channels of the disc may include or be in communication with one or more chambers. According to some aspects, at least one of the microfluidic channels may include at least one sample preparation chamber configured to extract genomic material present in the sample to be analyzed by the device, the genomic material comprising the target(s). Additionally or alternatively, the sample preparation chamber(s) may be configured to separate blood into components of plasma, serum, and cells. The microfluidic channels may include one or more reaction chambers, e.g., at least one reaction chamber that contains the substrate and the plurality of capture molecules. In some examples, at least one of the microfluidic channels may include two reaction chambers in communication with each other, wherein one of the two reaction chambers contains the plurality of capture molecules. The other reaction chamber may, for example, contain reagents for performing an amplification reaction with the at least one target, such as a polymerase chain reaction or an isothermal amplification reaction. Such reagents may comprise a plurality of oligonucleotide sequences as primers for amplification of the target(s). In some examples, the plurality of microfluidic channels may comprise a plurality of detection molecules, each detection molecule including a detectable label. The device may further comprise a power source and a detector, which may be configured to detect fluorescence, to collect optical images, or both. The microfluidic channels of the disc may comprise one or more valves, such as a burst valve, to control or regulate fluid flow through the channels.

The present disclosure also includes methods of detecting at least one target in a fluid sample using a microfluidic device, e.g., any of the devices described herein. According to some aspects, the method may comprise introducing the fluid sample into at least one microfluidic channel of a disc of the device, rotating the disc, such that the fluid sample flows radially outward through at least one microfluidic channel of the disc to combine with at least one capture molecule of a plurality of capture molecules, and detecting a signal from the disc indicative of a presence of at least one target in the sample. The target(s) may comprise, e.g., an oligonucleotide, a protein, a small molecule, or a combination thereof.

In some methods, the plurality of capture molecules may include at least one aptamer that binds to a target or targets in the fluid sample. Additionally or alternatively, the plurality of capture molecules may include at least one oligonucleotide that hybridizes to a target or targets in the fluid sample. According to some aspects of the present disclosure, the method of detecting one or more targets in the fluid sample may comprise amplifying the target(s) before detecting the target(s). Amplifying the target(s) may include performing a polymerase chain reaction or an isothermal amplification process. In some examples, amplifying the target(s) may include heating a chamber of the disc in which the at least one target(s) are amplified. The fluid sample may comprise any suitable biological fluid. For example, the fluid sample may comprise blood or may be obtained from blood. In some examples, the method may comprise extracting genomic material present in the fluid sample, wherein the genomic material comprises the target(s) to be detected.

The methods herein may comprise detecting a signal from the disc by detecting a fluorescence signal of a detection molecule attached to the target(s). According to some aspects of the present disclosure, detecting the signal from the disc may include analyzing the fluid sample with an optical reader to determine a presence or absence of the target(s) in the fluid sample. In at least one example, the target or targets may be biomarkers indicative of cancer, a cardiac disease, a respiratory disease, a neurological disease, an infectious disease, or antibiotic resistant genes.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments. Any features of an embodiment described herein (e.g., composition, medical device, method of treatment, etc.) may be combined with any other embodiment, and are encompassed by the present disclosure.

FIGS. 1A, 1B, and 1C show exemplary microfluidic discs, in accordance with some aspects of the present disclosure.

FIG. 2 shows an exemplary microfluidic disc, in accordance with some aspects of the present disclosure.

FIGS. 3A and 3B are schematics of capture molecules attached to a substrate, in accordance with some aspects of the present disclosure.

FIGS. 4, 5, and 6 show flowcharts of exemplary assays according to the present disclosure.

FIG. 7 shows exemplary components of a device, in accordance with some aspects of the present disclosure.

FIG. 8 shows an exemplary container of a device, in accordance with some aspects of the present disclosure.

### DETAILED DESCRIPTION

Embodiments of the present disclosure may address a need for alternative devices and methods for detecting targets or analytes of interest in a sample. Aspects of the present disclosure may offer certain advantages in screening patients, including large populations, for various health conditions.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Devices according to the present disclosure may allow for rapid analysis of a relatively small amount of sample to detect one or more targets of interest in the sample. In some aspects of the present disclosure, oligonucleotides may be used as probes or capture molecules for the specific and/or parallel capture of targets. The oligonucleotides may be coupled to a substrate, such as microbeads and/or a microarray. The devices and methods herein may be used to detect and/or quantify different types of target analytes, including, but not limited to, oligonucleotides, proteins, and small molecules. In some aspects, the use of microbeads may allow for separation of the target from reagents and/or other components of the sample, which may provide for a cleaner signal.

In some aspects, for example, oligonucleotides (natural or non-natural) may be used as probes or capture molecules to detect and/or quantify naturally-occurring oligonucleotides such as DNA and/or RNA in a sample (including, e.g., a complex sample, such as a raw sample). For example, the probe or capture oligonucleotide may be a single-stranded nucleic acid at least partially complementary to the target nucleic acid to provide for hybridization between the probe and target oligonucleotides. Further, for example, the probe or capture oligonucleotide may be an aptamer capable of binding to a specific target, such as a protein or small molecule.

The sample to be analyzed with the devices and methods herein may be obtained or derived from any subject of interest, including mammalian subjects such as, e.g., human subjects, e.g., patients. Mammalian subjects include both humans and non-humans. Exemplary mammals for which samples may be analyzed according to the methods herein include, but are not limited to, humans, non-human primates, canines, felines, murines, bovines, equines, and porcines.

According to some aspects of the present disclosure, the sample may comprise blood and/or other liquid samples of biological origin, solid tissue samples such as a biopsy specimen, tissue culture, or cells derived therefrom, and the progeny thereof. For example, the sample may be complex, e.g., a raw sample comprising multiple different types of cells, oligonucleotides, proteins, and/or other biological species. The sample may comprise a single cell or more than a single cell, e.g., a plurality of cells. Samples may include clinical samples, cells in culture, cell supernatants, and/or cell lysates. In at least one example, the sample may comprise a raw blood sample, or a blood sample that has been at least partially processed, e.g., blood plasma that has be separated from blood cells. In some aspects, a sample may be of cancerous origin, e.g., obtained from cancerous tissues. For example, the sample may be obtained from cancerous breast tissues.

The sample may be manipulated or processed by one or more procedures or treatment steps after their procurement from a subject. For example, a sample may be treated with one or more reagents, solubilized, and/or enriched for certain components. Enrichment of a sample may include, for example, concentrating one or more constituents of the sample to assist in detection, analysis, and/or identification of those constituent. In at least one example, a sample may be enriched for one or more target proteins and/or polynucleotides prior to exposing the sample to capture molecules for binding and detecting the target(s). The processing step(s) may be performed before and/or after the sample is introduced into the device for analysis.

In some examples, a raw sample may be processed to at least partially separate cellular material from liquid, e.g., separating blood cells from blood plasma in a raw blood sample. The liquid supernatant then may be introduced into the device for detection of analytes present in the liquid. In some examples, a raw biological sample may be processed to lyse cellular material chemically and/or by mechanical forces, and at least a portion of the lysed sample introduced into the device for analysis. In other aspects, a raw biological sample may be introduced into the device for separation and/or lysis of cellular material, e.g., in a microfluidic channel. For example, the configuration of the channel and/or beads (or other objects) disposed inside the channel may provide shearing forces or other mechanical force to rupture cellular membranes. Further, for example, the channel may include chemical and/or biochemical reagents capable of disrupting cellular walls in the sample upon contact with the sample. In yet additional examples, the device may be heated and/or ultrasound energy applied to induce lysis of the cellular material in a sample.

In some aspects of the present disclosure, the targets to be detected in a sample and/or the species used to detect the targets may comprise oligonucleotides. The term “oligonucleotide” includes, but is not limited to, nucleoside subunit polymers having contiguous subunits. The nucleoside subunits (e.g., adenosine, deoxyadenosine, guanosine, deoxyguanosine, 5-methyluridine, thymidine, uridine, deoxyuridine, cytidine, deoxycytidine, among other nucleosides) may be joined by a variety of inter-subunit linkages, including, but not limited to, phosphodiester, phosphotriester, methylphosphonate, P3′→N5′ phosphoramidate, N3′→P5′ phosphoramidate, N3′→P5′ thio-phosphoramidate, and phosphorothioate linkages. As used herein, the term “nucleoside” includes, but is not limited to, natural nucleosides, including, e.g., 2′-deoxy and 2′-hydroxyl forms, and analogs thereof. The term “analogs” in reference to nucleosides includes, but is not limited to, synthetic nucleosides having modified base moieties and/or modified sugar moieties. Such analogs may include, for example, synthetic nucleosides designed to enhance binding properties, e.g. stability, specificity, or the like.

The oligonucleotides herein may include one or more modifications to the sugar backbone (e.g., ribose or deoxyribose subunits), the sugar (e.g., 2′ substitutions), the nucleobases, and/or the 3′ and/or 5′ termini. In examples wherein the oligonucleotide moiety includes a plurality of inter-subunit linkages, each linkage may be formed using the same chemistry (e.g., the same linking group) or a mixture of different linkage chemistries (e.g., different types of linking groups) may be used. The term “polynucleotide” may be used interchangeably herein with the term “oligonucleotide.” The oligonucleotides may be natural and/or non-natural (synthetic). For example, the oligonucleotides may comprise DNA, RNA, microRNA (miRNA), synthetic nucleic acids, fragments thereof, or any combination thereof. In some aspects, the oligonucleotide may comprise one, two, or more than two non-natural nucleosides. In some aspects of the present disclosure, the oligonucleotides may comprise from 5 to 10,000 nucleotides, such as from 20 to 5,000 nucleotides, or from 100 to 1,000 nucleotides. In at least one example, the target oligonucleotide comprise a miRNA.

According to some aspects of the present disclosure, the target analytes to be detected in a sample may comprise biomarkers. The term “biomarker” generally refers to a chemical or biochemical indicator associated with one or more health conditions. A biomarker may include, but is not limited to, a molecule of interest or a portion of a molecule of interest that is to be detected and/or analyzed. Exemplary biomarkers include oligonucleotide sequences (e.g., DNA sequences and RNA sequences), small molecules, peptides, and proteins. Further, biomarkers according to the present disclosure may comprise fragments, splice variants, and/or full length peptides. Biomarkers according to the present disclosure include genetic markers, e.g., DNA sequences of an organism that may be useful in identifying characteristics of that organism. For example, the analyte may be a biomarker of a genetic disease, an environmental disease, a pathogen, or a resistance to an antibiotic. In some aspects, genetic markers associated with a disease or other health condition may include one or more alterations, variations, and/or mutations in a DNA sequence as compared to a DNA sequence that is not associated with the disease or other health condition. A biomarker or combination of biomarkers may be associated with a particular physical condition or health condition, e.g., a disease or disease state. For example, the biomarker(s) may be associated with breast cancer, e.g., late stage breast cancer.

The term “capture molecule” includes, but is not limited to, a molecule that is attached to, e.g., immobilized on, a surface for capturing a target present in a sample to be analyzed. As used herein, the term “immobilized” includes being immobilized, bound, and/or linked to a surface, such as a substrate. Exemplary substrates include, e.g., microarrays (including, e.g., slides, multi-well plates, and the walls or other inside surfaces of a detection device) and microbeads.

Capture molecules suitable for the present disclosure include, but are not limited to, RNA, DNA, aptamers, and protein-based aptamers. A capture molecule may bind to a target, e.g., a biomarker, in a sample to be analyzed. In some examples, the capture molecule may comprise an oligonucleotide, an aptamer, a chimeric structure comprising one or more oligonucleotide sequences, or an antibody.

In at least one example, the capture molecule comprises an oligonucleotide. The capture oligonucleotide may have a sequence at least partially or fully complementary to the sequence of a target oligonucleotide to be detected in the sample. For example, the capture oligonucleotide may comprise from 5 to 10,000 nucleotides complementary to the target, e.g., from 20 to 1,000 complementary nucleotides, from 50 to 500 complementary nucleotides, or from 100 to 300 complementary nucleotides. Thus, the capture oligonucleotide may hybridize to the target oligonucleotide to form a double-stranded nucleic acid attached to a substrate.

In some aspects of the present disclosure, a target may bind to a capture molecule, e.g., an aptamer. A molecule or other chemical/biochemical species may be said to exhibit “binding” if it reacts or associates more frequently, more rapidly, with greater duration and/or with greater affinity with one or more particular target(s) than with alternative substances (e.g., other targets or non-target species). For example, a capture molecule may “bind” to a target if it attaches to the target with greater affinity, avidity, more readily, and/or with greater duration than it attaches to other substances. In at least one example, the capture molecule may comprise an oligonucleotide that specifically or at least preferentially binds to a target (e.g., a biomarker) with greater affinity, avidity, more readily, and/or with greater duration than the oligonucleotide binds to other substances.

In at last one example, the capture molecule comprises an aptamer. The aptamer may comprise, for example, a single-stranded oligonucleotide (e.g., DNA or RNA) capable of binding to a target by structurally conforming to the target. The aptamer may be highly specific to, and form a strong bond with, a target.

Some methods according to the present disclosure may include size selection of oligonucleotides present in the sample, e.g., to produce target oligonucleotides of a desired size (e.g., nucleotide length). Size selection may be achieved, for example, with chemical reagents or enzymes to cleave oligonucleotides in the sample into shorter fragments suitable for capture and detection. Such fragments may have a length within a predetermined range, e.g., based on the properties of the chemical reagents or enzymes and reactivity with the sample.

The desired size of a target oligonucleotide may be selected based on the size and other properties of the corresponding capture molecule, e.g., for optimization of hybridization kinetics between the target and capture molecule. For capture molecules between 25 and 60 nucleotides in length, for example, target oligonucleotides between 50 and 200 nucleotides in length may provide for suitable hybridization. For capture molecules comprising thousands of nucleotides, larger-sized target oligonucleotides may be appropriate for binding or hybridization. In some aspects of the present disclosure, size selection of target oligonucleotides may provide uniformity of targets, e.g., to keep the kinetics of hybridization consistent. In at least some examples, size selection of oligonucleotides in a sample may not be performed. For example, miRNAs are typically short sequences (e.g., from 17 to 25 nucleotides in length), such that target miRNAs in a sample may combined with capture molecules without size selection.

Capture molecules may, or may not, be capable of binding solely to the target of interest. For example, a capture molecule may have one binding site, or a plurality of two or more binding sites. Capture molecules according to the present disclosure may be capable of binding to only one target (e.g., the capture molecule being specific to one particular target), to a select number of targets (e.g., the capture molecule being specific to two or more targets), or to a plurality of target and non-target species.

Further, a capture molecule that specifically or preferentially binds to a first target may or may not specifically or preferentially bind to a second target. In some aspects, for example, a capture molecule may bind to two or more targets, wherein the nature of the binding with each target may be about the same or may be different (e.g., the capture molecule having greater affinity for one target as compared to another target). As such, “binding” does not necessarily require (although it can include) exclusive binding. In some aspects, reference to “binding” may refer to preferential binding, e.g., a preference for reaction or association with one or more targets as compared to other species or substances. The concept of “binding” also is understood to include the concept of specificity, e.g., selective attachment between two species (e.g., a capture molecule and a target). Specific binding may be biochemically characterized as saturable (non-specific binding being non-saturable).

The capture molecule(s) may include, for example, one or more antibodies, peptides, proteins, or a combination thereof. Exemplary capture molecules suitable for the present disclosure include, but are not limited to, RNA, DNA, peptides, antibodies, aptamers, and protein-based aptamers. Exemplary capture molecules comprising antibodies are described in International Application No. PCT/US2016/030959 filed on May 5, 2016, incorporated by reference herein.

Linking of a capture molecule to a surface may be covalent or non-covalent. Linking capture molecules to a substrate may be achieved by any suitable method(s). For example, the substrate surface may be functionalized with one or more chemical functional groups, e.g., to be conjugated to capture molecules. Exemplary functional groups include, but are not limited to, amine, thiol, phosphate, alkyl, alkene, alkyne, arene, alcohol, ketone, aldehyde, carboxyl, and alkoxy groups.

In some aspects of the present disclosure, detection of a target may include binding the target to a detection molecule. For example, the detection molecules may comprise at least one detectable label (e.g., a chemical tag or probe molecule) that is detectable by an analytical technique such as optical detection, e.g., absorbance, fluorescence, chemiluminescence, or electrochemiluminescence. For example, the detectable label may comprise fluorescent agents, colorimetric agents, magnetic agents, or electrical agents, or any combination thereof. Fluorescent agents include, but are not limited to, quantum dots and fluorophores, e.g., including Alexa Fluor® 546 dye molecules and Alexa Fluor® 488 dye molecules produced by ThermoFisher Scientific, phycoerythrin (PE), and allophycynin (APC).

In at least some examples, the capture molecules may be attached to, or immobilized on, microbead surfaces. The term “microbead” as used herein includes, but is not limited to, particles having a generally curved shape. In at least one example, the microbeads may be spherical with a uniform diameter. Microbeads according to the present disclosure may be rigid, and may have a surface that is smooth or porous, or that includes both smooth portions and porous portions. A microbead may comprise one material or a combination of materials. The microbeads may have magnetic properties in some embodiments, e.g., the microbeads comprising a magnetic material or combination of materials.

According to some aspects of the present disclosure, the microbeads may have an average diameter between about 10 nm and about 100 μm, such as from about 50 nm to about 50 μm, from about 100 nm to about 10 μm, from about 100 nm to about 5 μm, from about 500 nm to about 5 μm, from about 100 nm to about 1 μm, from about 1 μm to about 50 μm, from about 5 μm to about 10 μm, or from about 10 μm to about 50 μm. For example, the microbeads may have an average diameter of about 10 nm, about 100 nm, about 500 nm, about 1 μm, about 5 μm, about 10 μm, about 50 μm, or about 100 μm.

In some examples, the capture molecules may be attached to, or immobilized on a surface to form a microarray. In some examples, a plurality of capture molecules specific to the same target may be grouped together in close proximity to one another, forming a “feature” of the microarray. Thus, for example, the microarray may include one or more features for detection of the same target. In some aspects, the microarray may include multiple features for detection of different types of targets, e.g., each feature comprising a plurality of capture molecules specific to a target. Each feature may range from about 10 μm to about 500 μm in cross-sectional size, such as from about 50 μm to about 100 μm, from about 75 μm to about 250 μm, or from about 100 μm to about 200 μm, e.g., a cross-sectional size of about 10 μm, about 50 μm, about 75 μm, about 100 μm, about 150 μm, about 200 μm, or about 250 μm. In some examples, the microarray may include 1 feature to 1 million features or more, such as from 5 to 10,000 features, from 10 to 1,000 features, or from 100 to 500 features. Further, for example, the microarray may include from 2 to 48 features, from 5 to 30 features, or from 8 to 25 features. The configuration of the microarray may be selected based on the number of features desired, the number and/or types of targets to be detected, and/or the available space on the surface of the substrate (e.g., the space available in the chamber or chambers of the disc to contain the microarray). In some examples, the features may be arranged in a regular pattern, such as in a rectangular, square, circular, triangular, or hexagonal pattern, or a combination thereof. For example, the microarray may have a grid-like configuration of 9 features (e.g., 3×3 square, or concentric circles of 5 and 4), 12 features (e.g., 3×4 rectangle), 16 features (e.g., 4×4 square), 20 features (e.g., 4×5 rectangle), or 25 features (e.g., 5×5 square). Each channel may include one microarray or a plurality of microarrays.

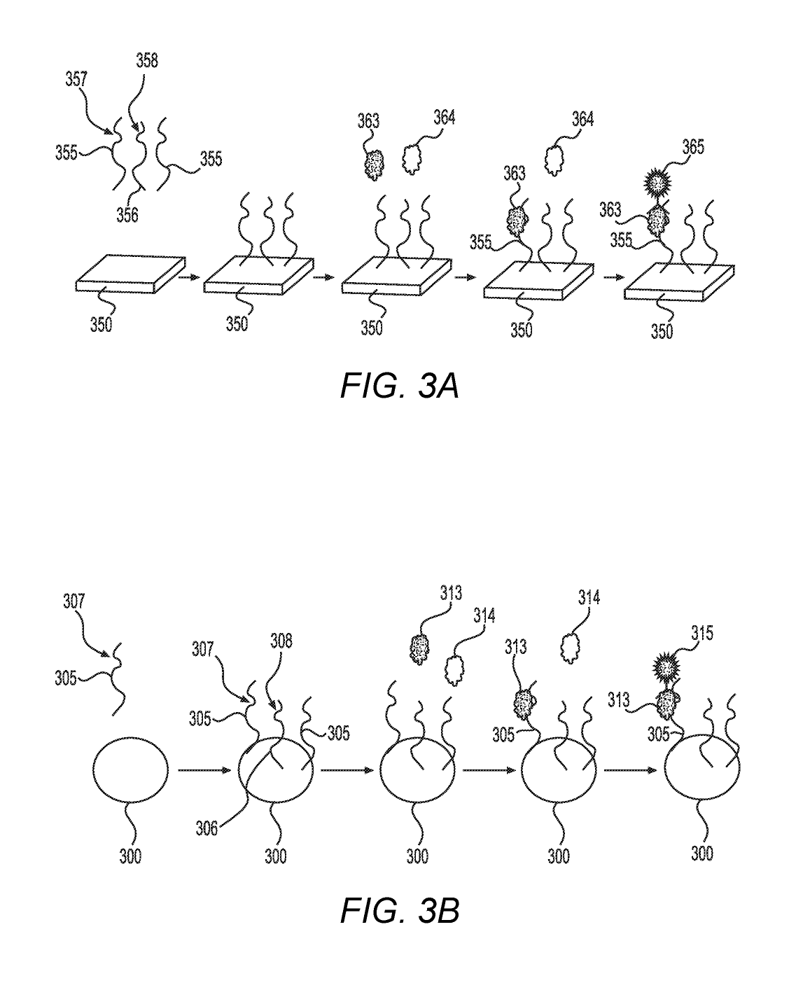

FIGS. 3A and 3B illustrate examples of capture molecules attached to substrates according to some aspects of the present disclosure.

FIG. 3A shows a portion of an exemplary substrate 350 comprising a microarray. The substrate 350 may be disposed in or incorporated into a detection device. For example, the substrate 350 may form a wall of the device (e.g., the wall of a chamber or of a microfluidic channel) or may comprise a microarray coupled to a wall of the device. As shown, two different types of capture molecules 355, 356 may be attached to the substrate 350. Each capture molecule 355, 356 may be covalently bonded to the surface via any suitable chemical linking group or entity of the capture molecule 355, 356 and/or of the surface of the substrate 350, such that a portion 357, 358 of the respective capture molecules 355, 356 is available for binding to or hybridization with a target. The portion 357, 358 of each capture molecule 355, 356 available for reacting with a target may be a binding site, such as a three-dimensional secondary structure of the capture molecule (e.g., in the case of an aptamer specific to a particular target), for example, or a length of the capture molecule, such as a nucleic acid sequence (e.g., in the case of oligonucleotides suitable for hybridization to a particular target).

When combined with a sample comprising a plurality of targets 363, 364, capture molecule 355 may selectively bind to or hybridize with target 363, but not target 364 (e.g., capture molecule 355 not being specific or complementary to target 364). Further, capture molecule 356 may not be specific or complementary to target 364, such that it does not bind or hybridize to target 364. A detection molecule 365 comprising a detectable tag (e.g., a fluorescence tag) specific to or complementary with the target 363 also may bind to or hybridize with the target 363 to allow for detection. Thus, target 363 may be captured for detection on the surface of the substrate 350 via its association with the immobilized capture molecule 355, whereas target 364 may not be detected.

FIG. 3B shows an exemplary microbead 300 as a substrate for use in some aspects of the present disclosure. As shown, two different types of capture molecules 305, 306 may be attached to the surface of the microbead 300. Each capture molecule 305, 306 may be covalently bonded to the surface via any suitable chemical linking group or entity of the capture molecule 305, 306 and/or of the surface of the microbead 300, such that a portion 307, 308 of the respective capture molecules 305, 306 is available for binding to or hybridization with a target. When combined with a sample comprising a plurality of targets 313, 314, capture molecule 305 may selectively bind to or hybridize with target 313 (e.g., forming a capture molecule-microbead/target complex), but not target 314. A detection molecule 315 comprising a detectable tag (e.g., a fluorescence tag) specific to or complementary with the target 313 also may bind to the target 313 to allow for detection. Further, capture molecule 306 may not be specific to or complementary with target 314, such that it does not bind to or hybridize with target 314. Thus, target 313 may be detected via its association with the microbead 300 and capture molecule 305, whereas target 314 may not be detected.

While FIGS. 3A and 3B illustrate examples wherein different types of capture molecules are attached to the same substrate surface (e.g., for capture and detection of different targets), in other examples the substrate may include only one type of capture molecule. For example, when microbeads are used as substrates, a plurality of set of microbeads may include the same type of capture molecule, such that the microbeads are specific to one target. Each microbead of the plurality of microbeads may have the same size, shape, and chemical composition as the other microbeads, or the plurality of microbeads may include at least one microbead having a different size, shape, and/or chemical composition than at least one other microbeads of the plurality of microbeads. Similarly, a surface of a chamber or channel of a microfluidic device may include a plurality of capture molecules of the same type, or the surface may be divided into two or more areas (e.g., defining multiple discrete features on the surface to form a microarray), each comprising a different type of capture molecule.

In some examples, the capture molecule(s) may be labeled, e.g., comprising at least one detectable label (e.g., a chemical tag or probe molecule). For example, the capture molecule(s) may comprise a label detectable by an analytical technique such as optical detection, e.g., fluorescence, chemiluminescence, or electrochemiluminscence. In some aspects, the capture molecule(s) may comprise a fluorescently-labeled oligonucleotide, antibody, or protein.

Some aspects of the present disclosure include analyzing a sample in a diagnostic assay to determine the presence or absence of one or more targets serving as biomarkers, and/or measuring the amount of one or more biomarkers in the sample. In at least one embodiment, the assay may comprise one or more capture molecules. For example, the assay may comprise a plurality or set of capture molecules. In some embodiments, the set may comprise at least two distinct capture molecules, wherein each distinct capture molecule may recognize or hybridize to a different target (e.g., a biomarker). The set of capture molecules may range from 2 to 1,000 or more capture molecules. In some embodiments, the set of capture molecules may comprise at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 100, 150, 200, 250, 500, 750, or 1,000 or more distinct capture molecules. For example, the set of capture molecules contained in a disc (e.g., coupled to microbeads in the same or different chambers, or attached to a surface to form one or more microarrays in the same or different chambers) may range from 2 to 1,000 distinct capture molecules, such as from 100 to 1,000, from 50 to 500, or from 2 to 100 distinct capture molecules.

In at last one example, the set of capture molecules includes 5, 6, or 7 distinct capture molecules each specific to or complementary with a different biomarker related to a particular health condition, such as, e.g. cancer, a cardiac disease, or a neurological disease. In another example, the set of capture molecules includes 12 distinct capture molecules each specific to or complementary with a different biomarker related to a particular health condition. In yet another example, the set of capture molecules includes between 20 and 1,000 or between 20 and 60 distinct capture molecules each specific to or complementary with a different biomarker related to a particular health condition or combination of health conditions. In some examples, one or more other target binding agents may be used, in addition to the capture molecules and capture molecule sets described herein.

The number and type(s) of capture molecules may depend on one or more of the following parameters: the contemplated uses and applications of the capture molecules, the complexity and composition of the sample, the binding affinity and/or specificity of the capture molecules, and/or the stability of the capture molecules. For example, the choice of capture molecules may depend on the targets to be detected in the sample. In some aspects, the capture molecule(s) may be specific to or complementary with one or more biomarkers of a set of biomarkers, e.g., a biomarker panel. For example, capture molecules may be specific to biomarkers associated with a particular health condition. In some aspects, each capture molecule may be specific to one biomarker of the panel.

In some examples, the targets to be detected may be biomarkers associated with breast cancer. For example, the biomarkers may include human estrogen receptor 2 (Her-2), matrix metallopeptidase-2 (MMP-2), cancer antigen 15-3 (CA 15-3), osteopontin (OPN), tumor protein p53 (p53), vascular endothelial growth factor (VEGF), cancer antigen 125 (CA 125), serum estrogen receptor (SER), or a combination thereof. Examples of sequence identifiers in the HUGO Gene Nomenclature Committee on-line database for such markers include, but are not limited to, Her-2 (X03363), MMP-2 (NM_004530), OPN (NM_001040058), p53 (NM_000546), VEGF (MGC70609), CA 125 (Q8WX17), SER (NP 000116.2), and CA 15-3 (NM_002456).

The devices and methods disclosed herein may be used for detection and/or diagnosis of conditions or diseases other than breast cancer. For example, sets of biomarkers may be chosen for other diseases such as, e.g., prostate cancer, ovarian cancer, heart disease, neurological disease, respiratory disease, and infectious diseases such as sexually transmitted diseases (STDs). Exemplary biomarkers for a prostate cancer panel (e.g., biomarkers useful in obtaining diagnostic information regarding prostate cancer) may include, but are not limited to, PSA. Exemplary biomarkers for an ovarian cancer panel (e.g., biomarkers useful in obtaining diagnostic information regarding ovarian cancer) may include, but are not limited to, CA 125. Exemplary biomarkers for a heart disease panel (e.g., biomarkers useful in obtaining diagnostic information regarding heart disease) may include, but are not limited to, troponin T, troponin I, CRP, homocysteine, myoglobin, and/or creatine kinase. Exemplary biomarkers for a respiratory disease panel (e.g., biomarkers useful in obtaining diagnostic information regarding respiratory disease) may include, but are not limited to, influenza A, influenza B, and respiratory syncytial virus (RSV). In some aspects of the present disclosure, the biomarkers of a panel may be associated with, or otherwise indicative of, pathogens (e.g., bacteria, viruses, parasites) linked to STDs and/or other infectious diseases. In some examples, the biomarkers of a panel may be associated with, or otherwise indicative of, antibiotic resistance to one or more pathogens.

According to some aspects of the present disclosure, “detect” may refer to identifying the presence, absence and/or amount of a target, such as an oligonucleotide, gene, small molecule, or protein, among other exemplary targets. Detection may be done visually and/or using any suitable device, such as, e.g., a scanner and/or detector. Further, any suitable analytical technique may be used for detection, including, but not limited to, optical techniques. Non-limiting examples of techniques that may be used in detection according to the present disclosure include absorbance, fluorescence, chemiluminescence, and electrochemiluminescence. In some aspects, detection may include use of charge coupled device (CCD) for imaging, e.g., a CCD camera.

The term “analyze” as used herein may include, but is not limited to, determining a value or a set of values associated with a given sample by a measurement. For example, analyzing according to some examples of the present disclosure may include measuring constituent expression levels in a sample and comparing the levels against constituent levels in a sample or set of samples from the same subject or other subject(s).

Devices suitable for various embodiments of the present disclosure may provide for point-of-care testing, e.g., to obtain diagnostic information for patient at or near the time and place of patient care. For example, the device may be portable and/or self-contained. Further, devices according to the present disclosure may be used to measure multiple targets (e.g., biomarkers) simultaneously, in a multiplex assay. In some aspects, the device may include microfluidic channels for performing a multiplex assay.

Microfluidic devices may improve kinetics of capture or detection, given small volumes used and the laminar flow involved in the processes. On a microfluidics platform, for example, a relatively small volume of sample (e.g., on the order of microliters (L)) may be sufficient to measure levels for a plurality of biomarkers. Moreover smaller volumes may make more efficient local heating and/or cooling processes, e.g., which may useful to speed up or otherwise facilitate thermally-induced reactions, such as the polymerase chain reaction (PCR).

In some aspects, the device may be a microfluidic-based immunoassay detection device comprising a microfluidic disc, a motor to control the spinning rate of the disc, and a detector such as an optical reader, e.g., to measure biomarkers. Microfluidic devices according to the present disclosure may include any of the features disclosed in U.S. Provisional Application No. 62/202,353, filed on Aug. 7, 2015, incorporated by reference herein.

Microfluidic discs of the present disclosure may comprise one or more channels that include a series of interconnected chambers, wherein reagents and sample may be mixed and/or moved from chamber to chamber by applying a centrifugal force. Thus, for example, the microfluidic disc may provide the channel(s) through which fluid flows and the chambers where reagents are stored and/or mixed with a sample added to the disc in a diagnostic assay. In general, the rotational speed of microfluidic disc may range from 50 to 20,000 revolutions per minute (RPM), such from 100 to 16,000 RPM, from 200 to 5,000 RPM, or from 500 to 10,000 RPM. The disc may rotate clockwise, counterclockwise, or both clockwise and counterclockwise alternately during an assay.

In some examples, the microfluidic disc may contain capture molecules attached to a mobile substrate, such as microbeads, which may undergo various processes (e.g., binding, separation, detection) of the assay by moving through microfluidic channels and chambers of the disc. In at least one example, the microfluidic disc may contain a plurality of microbeads conjugated with specific capture oligonucleotides. Additionally or alternatively, the microfluidic disc may contain capture molecules attached to a stationary substrate, such as a microarray, which may form a portion of, or may be coupled to, a microfluidic chamber or channel of the device. In at least one example, the microfluidic disc may contain a microarray having a plurality of oligonucleotides attached to the microarray surface. Reagents other than capture molecules attached to a substrate may be present in liquid, gel, or lyophilized form. When a portion of the reagents are lyophilized, the sample or sample component introduced into the microfluidic disc for analysis may reconstitute the lyophilized material(s).

In some examples, the microfluidic disc may contain oligonucleotides to serve as primers for a nucleic acid amplification reaction, and a suitable set of reagents for binding, detection and separation processes. For example, the oligonucleotides serving as primers may not be attached to a substrate, but instead may be pre-loaded into one or more chambers or channels of the microfluidic disc. Thus, upon introduction of a sample into the microfluidic disc, targets present in the sample may combine with the oligonucleotides to amplify or copy the target to facilitate detection of the target.

The channel or channels of the microfluidic disc may be any suitable shape including, e.g., round, trapezoidal, triangular, or other geometric shapes. Channels may be straight, curved, zig-zag, U-shaped, or other configurations, e.g., depending upon the application and function of the channel. Channel sizes may be selected based on one or more factors, such as the type(s) and/or number of targets (e.g., biomarkers) to be analyzed in a sample, the type(s) and/or number of capture molecules stored in the disc for binding with the target(s), the nature of binding between targets and capture molecules, among other factors. In some exemplary discs, the channels may be from about 0.01 microns to 5 millimeters deep and from 0.01 microns to about 5 millimeters wide. For example, the channels may range from about 0.05 microns to about 5 millimeters deep and from about 0.01 microns to about 1 centimeter or more in diameter. The fluid capacity of the channels may range from about 1 nanoliter to about 1 mL or more, depending upon the application.

Each channel may be in communication with an inlet for introduction of the sample to be analyzed. In general, an aliquot of the sample (such as, e.g., whole blood or other biological fluid) ranging from about 1 μL to about 300 μL or more (˜one to several drops) may be added to the inlet, such as from about 1 μL to about 280 μL, from about 1 μL to about 250 μL, from about 1 μL to about 220 μL, from about 1 μL to about 200 μL, from about 1 μL to about 180 μL, from about 1 μL to about 150 μL, from about 1 μL to about 120 μL, from about 1 μL to about 100 μL, from about 1 μL to about 80 μL, 1 μL to about 80 μL, from about 1 μL to about 40 μL, from about 1 μL to about 20 μL, from about 1 μL to about 6 μL, from about 20 μL to about 250 μL, from about 20 μL to about 200 μL, from about 50 μL to about 100 μL, from about 50 μL to about 250 μL, from about 100 μL to about 200 μL, from about 5 μL to about 80 μL, or from about 2 μL to about 5 μL. For example, an aliquot of sample of about 1 μL, about 2 μL, about 3 μL, about 4 μL, about 5 μL, about 6 μL about 20 μL, about 40 μL, about 60 μL, about 80 μL, about 100 μL, about 120 μL, about 150 μL, about 180 μL, about 200 μL, about 220 μL, about 240 μL, about 250 μL, about 280 μL, or about 300 μL may be used. As the disc rotates, the sample may flow through the channel(s), radially outward, by centrifugal force.

The microfluidic discs may be made of any material or combination of materials suitable for the assay. For example, the microfluidic disc may comprise one or more polymers or copolymers. Exemplary materials suitable for the microfluidic discs herein include, but are not limited to, polypropylene, polystyrene, polyethylene, acrylates such as poly(methyl methacrylate) (PMMA), cyclic olefin polymers (COP), cyclic olefin copolymers (COP), polydimethylsiloxane (PDMS), polyacrylamides, and combinations thereof.

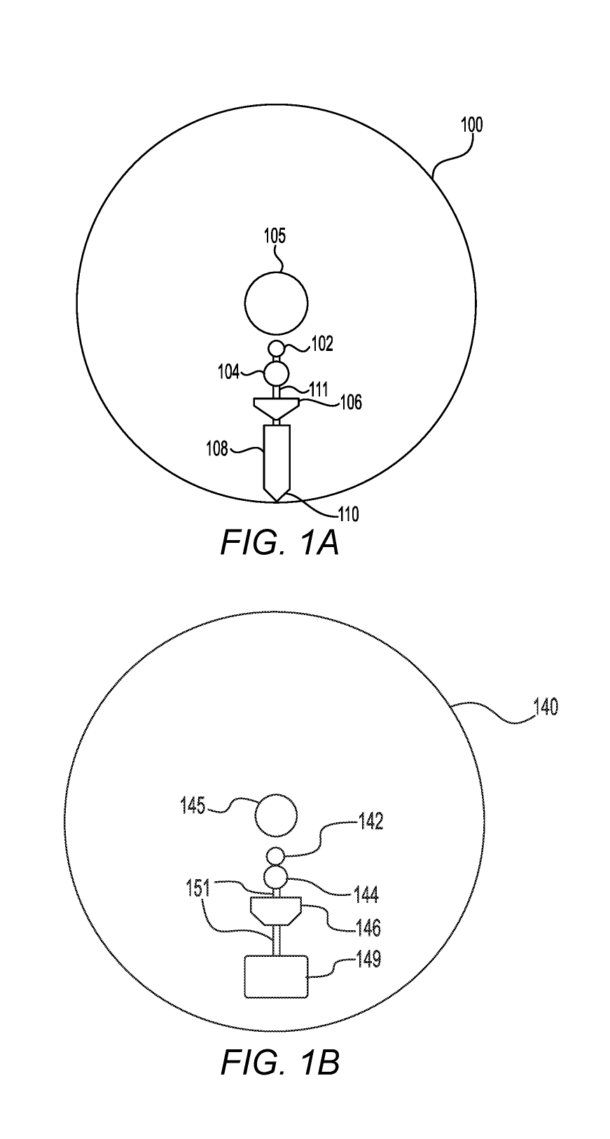

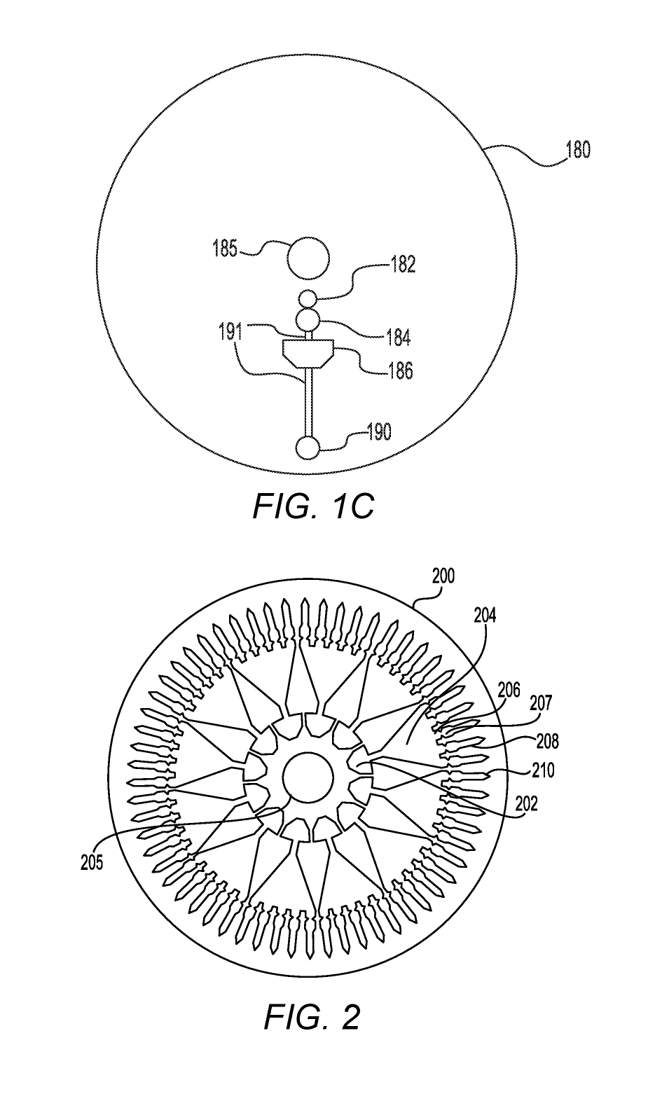

FIG. 1A shows an exemplary microfluidic disc 100 suitable for some assays according to the present disclosure in which microbeads are used. As shown, the disc 100 comprises one microfluidic channel that includes a series of interconnected chambers through which fluid may flow during an assay for illustrative purposes only. The disc 100 may include multiple channels disposed at different radial positions (see, e.g., FIG. 2). As shown, for example, the channel may include at least one sample inlet 102, at least one sample preparation chamber 104, at least one reaction chamber 106, at least one separation chamber 108, and at least one detection chamber 110. The disc 100 may include a central aperture 105, e.g., for coupling the disc 100 to a powered component to drive rotation of the disc 100 during an assay.

The combination, types, and sequence of chambers shown in FIG. 1A are illustrative only. The number and design of the chambers may be tailored to the particular targets being detected and the reagents used. For example, the disc 100 may not include a sample preparation chamber 104, e.g., if the assay does not include sample processing prior to being combined with reagents pre-loaded into the disc 100. Further, for example, the disc may not include a reaction chamber 106, e.g., if the assay does not include a reaction step prior to the capture of targets by a microarray substrate.

In an exemplary procedure, a sample, e.g., a blood sample that includes the targets of interest, is added to the sample inlet 102 of the microfluidic disc 100. The sample preparation chamber 104 may provide for pre-processing of the sample prior to mixing with reagents stored in the disc 100. For example, various components of the sample may be separated, e.g., via a filter or due to the configuration of the channel, such that only a portion of the original sample may flow through the channel to subsequent chambers for analysis. For example, the sample inlet 102 may be configured to separate whole blood into plasma, serum, and cell components. In some aspects of the present disclosure, the sample preparation chamber 104 may comprise reagents to assist in lysis and/or size separation of oligonucleotides present in the sample.

Depending on the type of sample processing used, the disc 100 may include additional sample preparation chambers 104 in sequence, e.g., for performing different processing steps as the sample flows through the channel. Further, in some examples, the disc 100 may include a separation or sedimentation chamber after the sample preparation chamber 104 for separating cellular material from the liquid supernatant (comprising the targets to be detected and analyzed). See, e.g., U.S. Provisional Application No. 62/202,353 filed on Aug. 7, 2015, incorporated by reference herein. If the assay does not include processing of the sample prior to being combined with reagents stored in the disc 100 (e.g., processing is not needed/desired, or processing is done before introducing the sample into the disc 100), the disc 100 may not include any sample preparation chambers 104.

In some aspects, the sample then may continue to flow through the channel to enter a reaction chamber 106 for combination with reagents pre-loaded into the reaction chamber 106. In some examples, the amount of sample component (e.g., blood plasma) mixed with reagents for analysis may generally range from about 1 μL to about 6 μL. For example, the amount of sample or sample component sufficient for a multiplex assay according to the present disclosure may range from about 2 μL to about 5 μL, e.g., an aliquot of sample of about 1 μL, about 2 μL, about 3 μL, about 4 μL, about 5 μL, or about 6 μL.

The term “reaction chamber” is intended to encompass a chamber in which various types of reactions and/or other interactions between target analytes and reagents pre-loaded into the disc may occur, and should not be construed as limited to a particular type of chemical reaction or interaction. For example, the reaction chamber 106 may include reagents designed for binding or hybridization to a target, and/or reagents designed for amplification of a target. Thus, for example, capture molecules attached to microbeads (see, e.g., FIG. 3B) and/or primer oligonucleotides for an amplification reaction may be included in the reaction chamber 106. In at least some examples, the reaction chamber 106 may be configured to control the amount of sample permitted to enter a subsequent chamber (e.g., the separation chamber 108), such that a portion of the reaction chamber 106 serves as a metering chamber. Additional examples of metering of the sample are discussed below.

If the assay includes multiple reaction steps, the disc 100 may include two or more reaction chambers 106 in sequence, each reaction chamber 106 including the appropriate reagents for the reaction. For example, the disc 100 may include two or more reaction chambers 106 for performing various steps of the assay, e.g., a first reaction chamber 106 containing a first set of reagents for amplification of a target, followed by a second reaction chamber 106 containing a second set of reagents for binding of the amplified target with capture molecules. The reaction chamber(s) 106 may be in communication with one or more waste chambers for receiving and storing excess sample and/or reagents.

After the reaction chamber(s) 106, the sample may continue to flow through the channel to enter the separation chamber 108. The separation chamber 108 may comprise microbeads serving as substrates, e.g., capture molecules being attached to the microbeads to form capture molecule-microbead/target complexes when combined with targets in a sample; see FIG. 3B. The separation chamber 108 may be configured to separate the microbeads from other reagents. For example, the separation chamber 108 may comprise a density medium, e.g., having a density less than that of the microbeads and greater than that of unbound reagents. Exemplary density media include Ficoll, although other materials having the appropriate density characteristics may be used. The microbeads may move through the density medium due to centrifugal forces from the rotating disc 100 to collect in a pellet in the detection chamber 110 while unbound reagents remain in the separation chamber 108. The pellet then may be analyzed by a detector to determine and analyze the presence and/or concentration of targets. The shapes of the separation chamber 108 and the detection chamber 110 may be designed to facilitate passage of the microbeads through the density medium and collection at the end of the channel. For example, the detection chamber 110 may have a generally tapered, V-shaped base, as shown in FIG. 1A, or any other suitable shape.

When the detection method is chemiluminescence or electrochemiluminscence, the disc 100 may include a suitable substrate/reagent pre-loaded into the detection chamber 110, such that the capture molecule-microbead/target complex may react with the substrate/reagent to generate light (e.g., ultraviolet, visible, or infrared light) for detection. In some aspects, the substrate/reagent may be present in a separate reservoir chamber, and may be added to the pellet in the same chamber where the pellet was generated (e.g., detection chamber 110) or in a separate chamber where the pellet and the substrate/reagent are combined.

In some examples, the disc 100 may include features to control fluid flow. For example, the disc 100 may include a valving system with relatively narrow channels, or burst valves, to regulate fluid flow. As shown in FIG. 1A, the disc 100 may include a valve 111 between a sample preparation chamber 104 and a reaction chamber 106. Additionally or alternatively, the disc 100 may include a valve 111 between a reaction chamber 106 and a separation chamber 108, between two reaction chambers 106, or between any other chambers discussed herein. The valve(s) 111 may provide resistance to fluid flow through the channels until enough force is provided to overcome such resistance. An example of force to overcome such resistance may include centrifugal force applied by spinning the disc at threshold speed. Each valve may be designed or adjusted to correspond to a particular rotational speed or speeds, e.g., such that different chambers may be selectively accessed to move the fluid at a desired time according to the operations of the device. In some aspects of the present disclosure, the disc 100 may comprise an air chamber or a pressure storage chamber, discussed in U.S. Provisional Application No. 62/202,353, filed on Aug. 7, 2015, incorporated by reference herein.

FIG. 1B shows an exemplary microfluidic disc 140 suitable for some assays according to the present disclosure, e.g., in which a microarray is used for detection of targets. The disc 140 is shown with one microfluidic channel for illustrative purposes only; the disc 140 may include multiple channels disposed at different radial positions (see, e.g., FIG. 2). The channel illustrated in FIG. 1B may include at least one sample inlet 142, at least one sample preparation chamber 144, at least one reaction chamber 146, and at least one array chamber 149.

The disc 140 may include any of the features of disc 100 discussed above. For example, the disc 140 may include a central aperture 145, e.g., for driving rotation of the disc 140 by a powered component, and one or more valves 151 between chambers to regulate fluid flow. Further, the sample inlet 142, sample preparation chamber 144, and reaction chamber 146 may include any of the features of the sample inlet 102, sample preparation chamber 104, and reaction chamber 106 of disc 100.

When a microarray is used as the substrate for capture molecules, the array chamber 149 may contain, or serve as, the microarray substrate. For example, capture molecules may be attached to the surface of the array chamber 149 for binding or hybridizing to targets present in the sample (see, e.g., FIG. 3A). As discussed above, the microarray may be designed for detection of one target (e.g., the microarray including capture molecules specific to a single target) or multiple, different targets (e.g., the microarray including a set of capture molecules, each capture molecule being specific to a different target and defining a different feature of the microarray). It should be noted that, in some assays, the targets to be detected may be bound or hybridized to capture molecules of a microarray without first reacting the sample with reagents. In such cases, the disc 140 may not include any reaction chambers 146, such that the sample inlet 142 or the sample preparation chamber 144 may lead into an array chamber 149.

In some aspects, the array chamber 149 may include detection molecules specific or complementary to the targets to assist in detection. The detection molecules may be combined with the targets before or after the targets are bound to the capture molecules of the microarray.

Once the targets have been bound to the capture molecules of the microarray, the array chamber 149 may be washed with a buffer solution, e.g., to clear away any unbound or unreacted reagents. The buffer solution may be introduced by activating one or more reservoir chambers in communication with the array chamber 149. The reservoir chambers may be activated, for example, by spinning the disc 140 at a threshold speed to open valves between the array chamber 149 and reservoir(s). After washing, the microarray may be scanned or imaged with a detector to analyze the targets in the sample. Such analysis may include identification and/or quantification of one or more query positions (e.g., target nucleotide sequence) in the targets. For example, targets bound to features of a microarray in the array chamber 149 may be imaged with a CCD camera to detect and measure the relative intensity of each feature of the microarray. The position of each feature may be associated with a specific capture molecule (e.g., an aptamer or oligonucleotide with known nucleic acid sequence), such that the positions of the features may be used to identify the targets detected. In some examples, the intensity of each feature may be used to determine the concentration of the target in the sample (e.g., based on a known relationship or correlation of intensity to target concentration). The detection may be performed in a single color mode or a dual color mode. A dual color mode may be useful, for example, in a comparative study to determine a relative copy number of genes, or an overexpression or under expression of specific genes or proteins in a control sample (e.g., healthy patient) as compared to an unknown sample.

FIG. 1C shows an exemplary microfluidic disc 180 suitable for some assays according to the present disclosure, such as assays that do not use microbeads or a microarray for detection of targets. The disc 180 is shown with one microfluidic channel for illustrative purposes only; the disc 180 may include multiple channels disposed at different radial positions (see, e.g., FIG. 2). The channel illustrated in FIG. 1C may include at least one sample inlet 182, at least one sample preparation chamber 184, at least one reaction chamber 186, and at least one amplification and detection chamber 190.

The disc 180 may include any of the features of discs 100 and/or 140 discussed above. For example, the disc 180 may include a central aperture 185, e.g., for driving rotation of the disc 180 by a powered component, and one or more valves 191 between chambers to regulate fluid flow. Further, the sample inlet 182, sample preparation chamber 184, and reaction chamber 186 may include any of the features of the sample inlets 102, 142, sample preparation chambers 104, 144, and reaction chambers 106, 146 of discs 100 and 140 discussed above.

The reaction chamber 186 and/or the amplification and detection chamber 190 may contain reagents for amplification of one or more target oligonucleotide(s) in the sample. The amplified targets then may be detected, e.g., without use of a substrate. In some examples, the amplification reaction may generate a byproduct (e.g., phosphate), which may be insoluble in the sample fluid. In such cases, the progression of the reaction may be monitored, e.g., by measuring turbidity in the amplification and detection chamber 190 over time. In other examples, the amplification and detection chamber 190 may contain capture molecules having specific, relatively short sequences that have a quencher and a detectable tag (e.g., a fluorescent tag) in proximity to each other. When the capture molecules are in presence of a complementary target sequence, they may hybridize to the target, and by doing so, the quencher and the detectable tag may be pushed apart. Once the detectable tag is apart from the quencher, the tag may be allowed to generate signal. For example, a fluorescent tag may be allowed to emit light.

As mentioned above, some chambers of a microfluidic disc may serve a metering function, e.g., to regulate an amount of sample that enters a subsequent chamber. Additionally or alternatively, the disc may include a separate metering chamber. In some examples, the microfluidic discs herein may comprise one or more metering chambers for dividing a sample between multiple subsequent chambers, e.g., to measure out the appropriate volume of sample for analysis as the sample flows radially outward during an assay. Referring to FIG. 1A, for example, the disc 100 may include a metering chamber that connects the sample preparation chamber 104 to multiple reaction chambers 106 at the same or approximately the same radius. Thus, after initial processing of a raw sample in the sample preparation chamber, the processed sample may be divided into two or more reaction chambers 106 each containing reagents specific to a different target. Each reaction chamber 106 then may be in communication with a different separation chamber 108 for detection and analysis of the different targets. Additionally or alternatively, the disc 100 may include a metering chamber between two reaction chambers 106, e.g., for dividing the sample following a first reaction into multiple aliquots prior to a second reaction. For example, the disc 100 may include a first reaction chamber 106 containing a set of reagents for amplification of one or more targets in the sample, wherein the first reaction chamber 106 leads into a metering chamber to divide the sample with the amplified target(s) between multiple second reaction chambers 106. Each second reaction chamber 106 may contain a set of reagents for binding the amplified target(s) with different types of capture molecules. Disc 140 and/or 180 also may include such metering chambers. Metering chambers are further discussed in connection to FIG. 2.

FIG. 2 shows an exemplary microfluidic disc 200 comprising a plurality of microfluidic channels according to some aspects of the present disclosure, wherein the disc 200 may be suitable for a multiplex assay. Each channel may include, or be in communication with, at least one sample inlet 202, at least one sample preparation chamber 204, at least one metering chamber 206, at least one reaction chamber 207, at least one separation chamber 208, and at least one detection chamber 210. For example, the channels may extend radially outward at regularly spaced intervals. In some aspects, the number of separation chambers 208 and detection chambers 210 (for detection of a target) may be greater than the number of sample inlets 202. As shown, for example, the disc 200 includes 12 sample inlets 202 each leading into a sample preparation chamber 204. Each of the 12 sample preparation chambers 204 is in communication with 5 metering chambers 206. Each metering chamber 206 leads into a reaction chamber 207 (e.g., where reagents may be pre-loaded into the disc 200), a separation chamber 208, and a detection chamber 210. Thus, the disc 200 may have a total of 60 channels, providing for analysis of 12 different samples, and at last 5 different targets per sample (e.g., if each reaction chamber 207 includes reagents specific to a different target). Each channel may include one or more valves similar to valves 111, 151, and 191 of FIGS. 1A-1C. The microfluidic disc 200 may include a central aperture 205 similar to apertures 105, 145, and 185 of discs 100, 140, and 180 in FIGS. 1A-IC. The disc 200 may include any of the features discussed above for the discs 100, 140, and/or 180 of FIGS. 1A-1C, such as an array chamber, multiple reaction chambers, and/or multiple metering chambers.

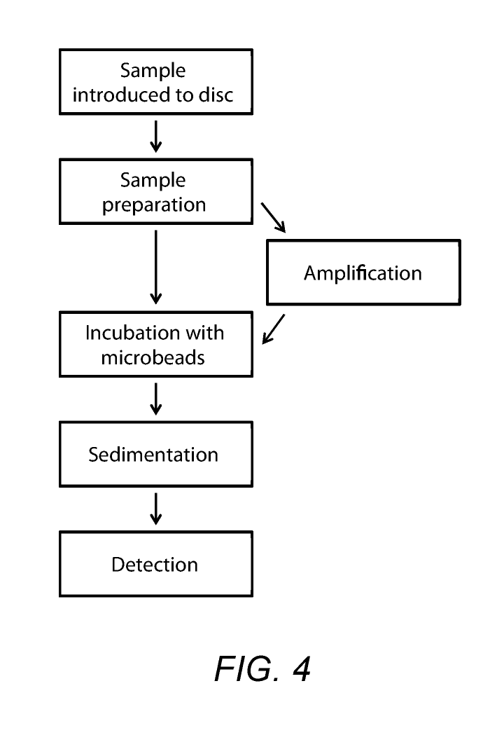

Microfluidic discs according to the present disclosure may be designed to perform different types of assays. FIGS. 4, 5, and 6 are flow diagrams outlining the steps of several exemplary assays using a microfluidic disc, which may include any of the features of microfluidic discs 100 and/or 200 discussed above. FIG. 4, for example, shows the steps of an assay that may be performed in a microfluidic disc comprising at least an inlet, a sample preparation chamber, one or more reaction chambers, a separation chamber, and a detection chamber. Oligonucleotides having a known nucleotide sequence complementary to the sequence of a target of interest may be attached to microbeads, which may be pre-loaded into the reaction chamber.

In the assay, a sample such as a raw blood sample may be introduced into the inlet of the disc, e.g., with a pipet or other suitable injection device. Upon rotation of the disc, fluid may flow through the channels, radially outward, from the inlet to the sample preparation chamber for lysis of the cellular material. The sample then may proceed to a reaction chamber for binding of the targets to capture molecules attached to the microbeads, and to detection molecules. The binding may occur during an incubation period. The microbead/target complexes thus formed in the sample may proceed to the separation chamber comprising a density medium to separate the complexes from unbound reagents. Finally, the complexes may proceed to the detection chamber proximate the edge of the disc to collect as a pellet for detection.

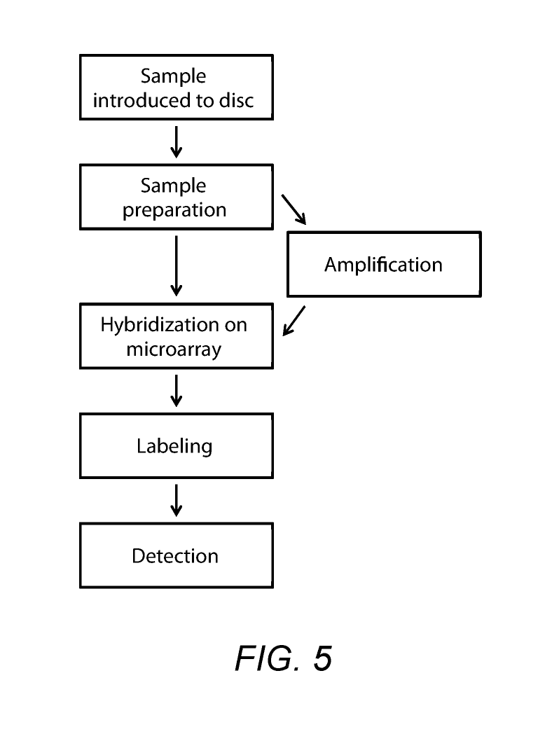

FIG. 5 shows the steps of an assay with some steps similar to those of FIG. 4, however a microarray may be used in place of microbeads for binding to the target(s). For example, the type of assay outlined in FIG. 5 may be performed in a microfluidic disc comprising at least an inlet, a sample preparation chamber, one or more reaction chambers, and an array chamber. Oligonucleotides having a known nucleotide sequence complementary to the sequence of a target of interest may be attached to the microarray in the array chamber. The array chamber also may include detection molecules specific to the target of interest to allow for labeling of the targets bound to the microarray, followed by detection.

As shown in FIGS. 4 and 5, some assays may include amplification of one or more target oligonucleotide(s) in the sample before binding to capture molecules pre-loaded into the microfluidic disc and/or to facilitate detection of the targets. In order to amplify the detection of specific sequences present in sample, the assay may include a step to amplify the target genomic material. For example, the assay may include a nucleic acid amplification technique, where one or multiple regions of each target oligonucleotide present in the sample may be amplified by PCR or isothermal techniques. In some aspects, for example, a reaction chamber may be exposed to multiple temperature gradients to generate a PCR-like reaction or an isothermal amplification reaction. The temperature may be controlled locally at the reaction chambers and/or within the device to obtain the desired gradient. In at least one example, RNA present in the sample may be treated with reverse transcriptase enzymes to obtain the relative complementary DNA (cDNA). Further, for example, the assay may include amplification by use of specific or non-specific primers to obtain an enrichment of specific regions of interest of the target, or to obtain a whole genome amplification. Such amplification processes may be performed in the presence of and/or may include non-natural nucleotides or nucleosides to achieve specific biophysical properties in the oligonucleotides, such as melting temperature (Tm).

Amplification of target sequences according to the present disclosure may be performed with and/or without a bias. For example, some assays may include amplification without a bias, such as a Whole Genome Amplification. For example, a relatively small amount of target genomic material (e.g., about 10 ng to about 50 ng) in a sample may be replicated to obtain a larger quantity of targets, more suitable for detection. During amplification, a detectable label may or may not be added to the target(s) to be detected.

In other examples, the amplification may be biased, such as by using of ad hoc primers to amplify specific sequences of interest of the targets. Biased amplification reactions may be useful, for example, to detect the presence of a specific gene, a set of genes, and/or a portion of a gene. For example, an assay may be performed to determine whether a sample contains a specific type of bacteria and if the bacteria is resistant to a specific antibiotic agent. The assay may include specific primers designed to amplify the portion of the genome of the bacteria specific for the identification of that bacterial species, and primers for the amplification of the bacterial gene indicative of a resistance to a given class of antibiotic agents.

The following examples (1) and (2) describe assays of the general type shown in FIG. 4.

(1) Nucleic Acid Probe on Microbeads for Detection of Oligonucleotides

In at least one example, oligonucleotides (comprising natural and/or non-natural nucleotides) of known sequence(s) and of variable length (e.g., comprising from 5 to 10,000 nucleotides, such as from 20 to 5,000 nucleotides) may be immobilized on the surface of microbeads with a diameter between about 10 nm and 100 μm, such as between 100 nm and 10 μm. The microbeads may be pre-loaded into a microfluidic disc.

A sample comprising genomic material may be introduced into an inlet of the disc. The disc may be then spun at a speed between 100 and 16,000 RPM, such as between 500 and 10,000 RPM. Centrifugal force generated by the rotation of the disc may cause the sample to flow into a sample preparation chamber, where the sample may contact reagents pre-loaded into the sample preparation chamber designed to extract the genomic material from the sample, e.g., via chemical or physical lysis.

The disc then may be spun at a speed between 100 and 16,000 RPM, such as between 500 and 10,000 RPM, in both directions, e.g., alternating clockwise and counterclockwise, for a time between 30 seconds and 30 minutes. For example, the disc may be spun clockwise and counterclockwise for less than 1 second each, about 1 second each, about 10 seconds each, about 1 minute each, about 5 minutes each, or about 10 minutes each, repeating up to a total time between about 30 seconds and about 30 minutes. The clockwise and counterclockwise rotations need not be identical in duration, e.g., the clockwise rotation being longer than the counterclockwise rotation. Further, successive rotations may have different durations.

After the extraction/lysis step the disc may be spun at a speed between 100 and 16,000 RPM, such as between 500 and 10,000 RPM and the processed sample transferred to a separation chamber to separate solid cellular material from the liquid supernatant. For example, the disc may be spun at a speed between 100 and 16,000 RPM, such as between 500 and 10,000 RPM to separate the cells from the rest of the sample. Then, the liquid supernatant of the sample comprising the genomic material, free of cells, may be transferred into a first metering chamber where the sample may be divided into multiple reaction chambers (first reaction chambers) of identical or different volumes. In some aspects, the transfer of the supernatant may be achieved with the activation of an air chamber, e.g., by appropriate control of the spinning rate of the disc.

The first metering chamber may be connected to each of the first reaction chambers via a hydrophobic valve. The disc then may be spun at a speed between 100 and 16,000 RPM, such as between 500 and 10,000 RPM to move the sample from the first metering chamber into the first reaction chambers. Within the first reaction chambers the genomic material present in the sample may interact with preloaded reagents (which may include, e.g., primers, enzymes, buffer solution, fluorescent dyes, among other suitable reagents) present in the first reaction chambers. Each first reaction chamber may include reagents specific for one or multiple query positions (e.g., target nucleotide sequences) in the genomic material of interest.

The first reaction chambers may be exposed to multiple temperature gradients to generate a PCR-like reaction or an isothermal amplification reaction as discussed above. In some aspects, the oligonucleotide products of the reactions described above may be subject to a lysis step to control the size of the oligonucleotides, e.g., to comprise from 50 to 10,000 nucleotides.

After the reaction is completed, the disc may be spun at a speed between 100 and 16,000 RPM, such as between 500 and 10,000 RPM to transfer the reacted sample into a second metering chamber, where the sample may be divided into multiple reaction chambers (second reaction chambers) of identical or different volumes. The second metering chamber may be connected to each of the second reaction chambers via a hydrophobic valve. The disc may be spun at a speed between 100 and 16,000 RPM, such as between 500 and 10,000 RPM to move the product of the reaction into the second reaction chambers, which may be pre-loaded with microbeads conjugated to capture oligonucleotides having sequences at least partially complementary to the sequences of the targets. Thus, for example, the target oligonucleotides may hybridize to the capture oligonucleotides to tether the targets to the microbeads. The second reaction chambers also may include detection molecules having a detectable label or tag, such as a fluorescent tag, wherein the detection molecules may bind to the hybridized target/capture molecule/microbead complex.

The disc may be spun in both directions, e.g., at a speed between 100 and 16,000 RPM, such as between 500 and 10,000 RPM for a time between 5 minutes and 24 hrs. During this time the second reaction chambers may be held at a constant temperature or at a gradient of different temperatures, e.g., between 15° C. and 95° C.

After the hybridization reaction step is completed the disc may be spun at a speed between 100 and 16,000 RPM, such as between 500 and 10,000 RPM to move the microbeads into a detection chamber at least partially filled or completely filled with density media. The density media may be chosen to have a relative density lower than the microbeads and higher than the reagents and unreacted components in the sample.

The disc may be spun at a speed between 100 and 16,000 RPM, such as between 500 and 10,000 RPM to allow the microbeads to settle at the bottom of the detection chamber in the form of a pellet. The pellet so generated may be detected by fluorescence or other methods depending on the nature of the detectable label.

(2) Aptameric Probes on Microbeads for Detection of Target Analytes

In at least one example, oligonucleotides (comprising natural or non-natural nucleotides) of known sequence(s) and of variable length (e.g., comprising from 5 to 10,000 nucleotides, such as from 20 to 1,000 nucleotides) may be immobilized on the surface of microbeads with a diameter between about 10 nm and 100 μm, such as between 100 nm and 10 μm. The microbeads may be pre-loaded into a microfluidic disc.

The sequences of the oligonucleotides (aptamers) may be selected to have a strong and specific binding interaction with a large set of molecularly and/or clinically relevant entities, including, but not limited to, proteins or small molecules.

A sample comprising the material of interest then may be introduced into the microfluidic disc. The disc then may be spun at a speed between 100 and 16,000 RPM, such as between 500 and 10,000 RPM to move the sample into the sample preparation chamber. The sample preparation chamber may contain reagents for separating out cellular material, e.g., via chemical or physical lysis.

The disc may be spun at a speed between 100 and 16,000 RPM, such as between 500 and 10,000 RPM to separate the cells from the rest of the sample. In at least one example, the disc may be spun in both directions, e.g., alternating clockwise and counterclockwise, at a speed between 100 and 16,000 RPM, such as between 500 and 10,000 RPM to activate lysis of cells in the sample and/or to separate the cellular material.

After the separation step is completed, the disc may be spun at a speed between 100 and 16,000 RPM, such as between 500 and 10,000 RPM and the sample may be transferred into a metering chamber connected via a hydrophobic valve to a series of reaction chambers. In some aspects, the transfer of the supernatant may be achieved with the activation of an air chamber, e.g., by appropriate control of the spinning rate of the disc.

The disc may be then spun at a speed between 100 and 16,000 RPM, such as between 500 and 10,000 RPM to move the sample from the metering chamber to the reaction chambers. Each reaction chamber may be pre-loaded with the aptamers attached to microbeads, as well as detection molecules capable of binding to the targets. Exemplary detection molecules may include, but are not limited to, fluorescently-labeled antibodies, fluorescently-labeled proteins, and other fluorescently-labeled molecules. Detectable tags other than fluorescent tags or labels may be used, however.

The disc then may be spun in both directions, e.g., alternating clockwise and counterclockwise, at a speed between 100 and 16,000 RPM, such as between 500 and 10,000 RPM for a total time between 5 minutes and 24 hours, such as between 5 minutes and 1 hour. During this time the reaction chambers may be held at a constant temperature and/or at a gradient of different temperatures, e.g., between 15° C. and 95° C.

After the reaction with the microbead-bound apatamers and detection molecules is completed, the disc may be spun at a speed between 100 and 16,000 RPM, such as between 500 and 10,000 RPM and the sample transferred into a detection chamber partially filled or completely filled with density media. The density media may be chosen to have a relative density lower than the microbeads and higher than the reagents and unreacted components in the sample.

The disc may be spun at a speed between 100 and 16,000 RPM, such as between 500 and 10,000 RPM to allow the beads to settle at the bottom of the detection chamber in the form of a pellet. The pellet so generated may be detected by fluorescence or other methods depending on the nature of the detectable label.

The following examples (3) and (4) describe assays of the general type shown in FIG. 5.

(3) Nucleic Acid Detection by Hybridization

In at least one example, oligonucleotides (comprising natural and/or non-natural nucleotides) of known sequence(s) and of variable length (e.g., comprising from 5 to 10,000 nucleotides, such as from 20 and 5,000 nucleotides) may be immobilized on a surface, e.g., a microarray. The microarray may include a plurality of oligonucleotide capture molecules (nucleic acid probes) distributed in a predetermined pattern, e.g., a configuration of features, wherein a collection of capture molecules specific to a target defines each feature on the microarray surface. Thus, the topological distribution of the probes and their sequences may be known and organized in a pre-determined manner. The size of each feature on the surface may range from about 1 μm to about 500 μm, such as from about 50 μm to about 150 μm, e.g., about 100 μm. In some examples, the total number of features on the microarray may range from 10 to 100 million, such as from 50 to 100,000 or 100 to 10,000 features.

A sample comprising the genomic material of interest may be introduced into an inlet of the disc. The disc then may be spun at a speed between 100 and 16,000 RPM, such as between 500 and 10,000 RPM. Centrifugal force generated by the rotation of the disc may cause the sample to flow into a sample preparation chamber, where the sample may contact reagents pre-loaded into the sample preparation chamber designed to extract the genomic material from the sample, e.g., via chemical or physical lysis.

The disc then may be spun at a speed between 100 and 16,000 RPM, such as between 500 and 10,000 RPM, in both directions, e.g., alternating clockwise and counterclockwise, for a time between 30 seconds and 30 minutes. For example, the disc may be spun clockwise and counterclockwise for 10 seconds each, 1 minute each, 5 minutes each, or 10 minutes each, repeating up to a total time between 30 seconds and 30 minutes.

After the extraction/lysis step, the disc may be spun at a speed between 100 and 16,000 RPM, such as between 500 and 10,000 RPM and the processed sample transferred to a separation chamber to separate solid cellular material from the liquid supernatant. For example, the disc may be spun at a speed between 100 and 16,000 RPM, such as between 500 and 10,000 RPM to separate the cells from the rest of the sample. Then, the liquid supernatant of the sample comprising the genomic material, free of cells, may be transferred into a metering chamber connected via a hydrophobic valve to multiple reaction chambers. In some aspects, the transfer of the supernatant may be achieved with the activation of an air chamber, e.g., by appropriate control of the spinning rate of the disc.

The disc then may be spun at a speed between 100 and 16,000 RPM, such as between 500 and 10,000 RPM to move the sample from the metering chamber into the reaction chambers. Within the reaction chambers, the genomic material present in the sample may interact with pre-loaded reagents (which may include, e.g., primers, enzymes, buffer solution, fluorescent dyes, among other suitable reagents) present in the reaction chambers. Each reaction chamber may include reagents specific for one or multiple query positions (e.g., target nucleotide sequences) in the genomic material of interest.

The reaction chambers may be exposed to multiple temperature gradients to generate a PCR-like reaction or an isothermal amplification reaction as discussed above. In some aspects, the oligonucleotide products of the reactions described above may be subject to a lysis step to control the size of the oligonucleotides, e.g., to comprise from 10 to 10,000 nucleotides.

After the reaction is completed the disc may be spun at a speed between 100 and 16,000 RPM, such as between 500 and 10,000 RPM to move the product of the reactions into respective array chambers in communication with the reaction chambers. Each array chamber may include the same or different type of microarray, and also may include detection reagents specific to the targets to be detected.

The disc may be spun in both directions, e.g., alternating clockwise and counterclockwise, at a speed between 100 and 16,000 RPM, such as between 500 and 10,000 RPM for a total time between 1 minute and 24 hours. During this time the array chambers may be held at a constant temperature and/or at a gradient of different temperatures, e.g., between 15° C. and 95° C.

After the targets bind to the microarrays and detection molecules in the array chambers, the disc may be spun at a speed between 100 and 16,000 RPM, such as between 500 and 10,000 RPM to move the sample (comprising unbound components) from the array chambers to respective waste chambers or a common waste chamber. The array chamber then may be washed with a buffer solution by activating one or more reservoir chambers in communication with the array chambers. For example, to open valve between the reservoirs and array chambers, the disc may be spun at a speed between 100 and 16,000 RPM, such as between 500 and 10,000 RPM. After washing, the microarrays within the array chambers may be scanned or imaged to provide analyze and/or quantify the query positions (nucleotide sequences) of interest in the genomic material of the sample.

(4) Detecting Small Molecules or Proteins by Hybridization on Aptameric Arrays

In at least one example, oligonucleotides (comprising natural and/or non-natural nucleotides) of known sequences and of variable length (e.g., comprising from 5 to 5,000 nucleotides, such as from 20 and 1,000 nucleotides) may be immobilized on a surface, e.g., a microarray. Similar to the example (3) above, the topological distributions of oligonucleotide capture molecules (nucleic acid probes) and their sequences may be known and organized on the microarray in a pre-determined manner. The size of each feature on the surface may range from about 1 μm to about 500 μm, such as from about 50 μm to about 150 μm, e.g., about 100 μm. In some examples, the total number of features on the microarray may range from 10 to 100 million, such as from 50 to 100,000.

The sequences of the oligonucleotides (aptamers) may be selected to have a strong and/or specific binding interaction with a relatively large set of molecularly and/or clinically relevant entities, including, but not limited to, proteins or small molecules.

A sample comprising the genomic material of interest may be introduced into an inlet of the disc. The disc then may be spun at a speed between 100 and 16,000 RPM, such as between 500 and 10,000 RPM to move the sample into the sample preparation chamber where the sample is in contact with pre-loaded reagents.

Centrifugal force generated by the rotation of the disc may cause the sample to flow into a sample preparation chamber, where the sample may contact reagents pre-loaded into the sample preparation chamber designed to extract the genomic material from the sample, e.g., via chemical or physical lysis. The disc may be spun at a speed between 100 and 16,000 RPM, such as between 500 and 10,000 RPM to separate the cells from the rest of the sample.

In some examples, the disc may be spun in both directions, e.g., alternating clockwise and counterclockwise, at a speed between 100 and 16,000 RPM, such as between 500 and 10,000 RPM to activate the lysis cells present in the sample.

After the extraction/lysis step, the disc may be spun at a speed between 100 and 16,000 RPM, such as between 500 and 10,000 RPM to move the processed sample into a metering chamber connected via a hydrophobic valve to a series of array chambers. In some aspects, the transfer of the supernatant may be achieved with the activation of an air chamber, e.g., by appropriate control of the spinning rate of the disc.

The disc may be spun at a speed between 100 and 16,000 RPM, such as between 500 and 10,000 RPM to move the sample from the metering chamber into the array chamber. The array chambers may be pre-loaded with detection reagents, e.g., detection molecules. Exemplary detection molecules may include, but are not limited to, fluorescently-labeled antibodies, fluorescently-labeled proteins, and other fluorescently-labeled molecules. Detectable tags other than fluorescent tags or labels may be used, however.

The disc then may be spun in both directions, e.g., alternating clockwise and counterclockwise, at a speed between 100 and 16,000 RPM, such as between 500 and 10,000 RPM for a total time between 5 minutes and 24 hours, such as between 5 minutes and 1 hour. During this time the array chambers may be held at a constant temperature and/or at a gradient of different temperatures, e.g., between 15° C. and 95° C.

After the targets bind to the microarrays and detection molecules in the array chambers, the disc may be spun at a speed between 100 and 16,000 RPM, such as between 500 and 10,000 RPM to move the sample (comprising unbound components) from the array chambers to respective waste chambers or a common waste chamber. The array chamber then may be washed with a buffer solution by activating one or more reservoir chambers in communication with the array chambers. For example, to open valve between the reservoirs and array chambers, the disc may be spun at a speed between 100 and 16,000 RPM, such as between 500 and 10,000 RPM. After washing, the microarrays within the array chambers may be scanned or imaged to provide analyze and/or quantify the query positions (nucleotide sequences) of interest in the genomic material of the sample.

(5) Nucleic Acid Amplification Detection

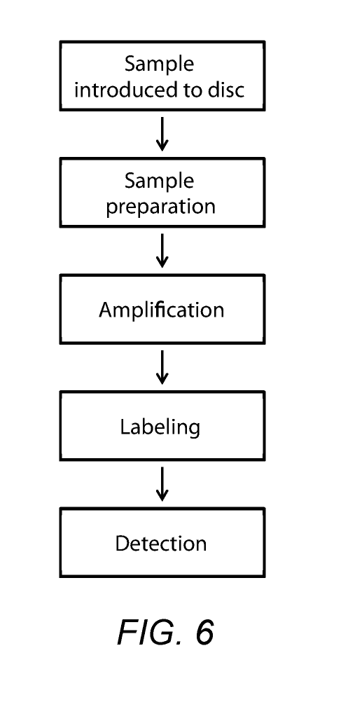

FIG. 6 shows yet another type of assay that may be performed on a microfluidic disc in accordance with some aspects of the present disclosure. In this type of assay, targets in the sample may be amplified as discussed above (e.g., via a PCR-like reaction or an isothermal amplification reaction), and then reacted with detection molecules to allow for detection of the targets. In contrast to examples (1)-(4) discussed above, the assay may not include capture molecules attached to a substrate (e.g., microbeads or a microarray) to bind the targets to the substrate for detection. Instead, the targets bound to the detection molecules may be localized or concentrated in a detection chamber (or amplification and detection chamber) for detection, e.g., by optical detection or another suitable detection technique appropriate for the tag of the detection molecule. Detection may include monitoring the product of an amplification reaction or observation of a detectable tag upon separation from a quencher as discussed above.

In at least one example, oligonucleotides (comprising natural or non-natural nucleotides) of known sequences and of variable length (e.g., comprising from 5 to 500 nucleotides) may be used as primers for enzyme-catalyzed amplification of specific target sequences in oligonucleotides of interest in the sample.

In the assay, a sample comprising the genomic material of interest may be introduced into an inlet of the disc. The disc then may be spun at a speed between 100 and 16,000 RPM, such as between 500 and 10,000 RPM. Centrifugal force generated by the rotation of the disc may cause the sample to flow into a sample preparation chamber, where the sample may contact reagents pre-loaded into the sample preparation chamber designed to extract the genomic material from the sample, e.g., via chemical or physical lysis.

After the extraction/lysis step, the disc may be spun at a speed between 100 and 16,000 RPM, such as between 500 and 10,000 RPM and the processed sample transferred to a separation chamber to separate solid cellular material from the liquid supernatant. For example, the disc may be spun at a speed between 100 and 16,000 RPM, such as between 500 and 10,000 RPM to separate the cells from the rest of the sample. Then, the liquid supernatant of the sample comprising the genomic material, free of cells, may be transferred into a metering chamber connected via a hydrophobic valve to multiple reaction chambers of identical or different volumes. In some aspects, the transfer of the supernatant may be achieved with the activation of an air chamber, e.g., by appropriate control of the spinning rate of the disc.

The disc then may be spun at a speed between 100 and 16,000 RPM, such as between 500 and 10,000 RPM to move the sample from the metering chamber into the reaction chambers. Within the reaction chambers, the genomic material present in the sample may interact with pre-loaded reagents (which may include, e.g., primers, enzymes, buffer solution, among other suitable reagents) present in the reaction chambers. Each reaction chamber may include reagents specific for one or multiple query positions (e.g., target nucleotide sequences) in the genomic material of interest.

The reaction chambers may be exposed to multiple temperature gradients to generate a PCR-like reaction or an isothermal amplification reaction as discussed above. The temperature may be controlled locally at the reaction chambers and/or within the device to obtain the temperature gradient desired.

In some examples, the reagents for detection may be pre-loaded in the reaction chambers, such that the progress of the amplification reaction may be monitored in real time and/or after completion of the amplification process. Additionally or alternatively, detection may be performed in separate detection chambers in communication with a corresponding reaction chamber. For example, after the amplification reaction is completed the disc may be spun at a speed between 100 and 16,000 RPM, such as between 500 and 10,000 RPM to move the products of the reaction into the detection chambers. In the detection chambers, the amplified products may react with the detection reagents (e.g., detection molecules such as intercalating dyes, fluorescently tagged probes, among other suitable detection molecules) to allow for measurement and quantification of the amplification of targets, and to detect the presence of the query positions (e.g., target nucleotide sequences) in the genomic material of the sample.

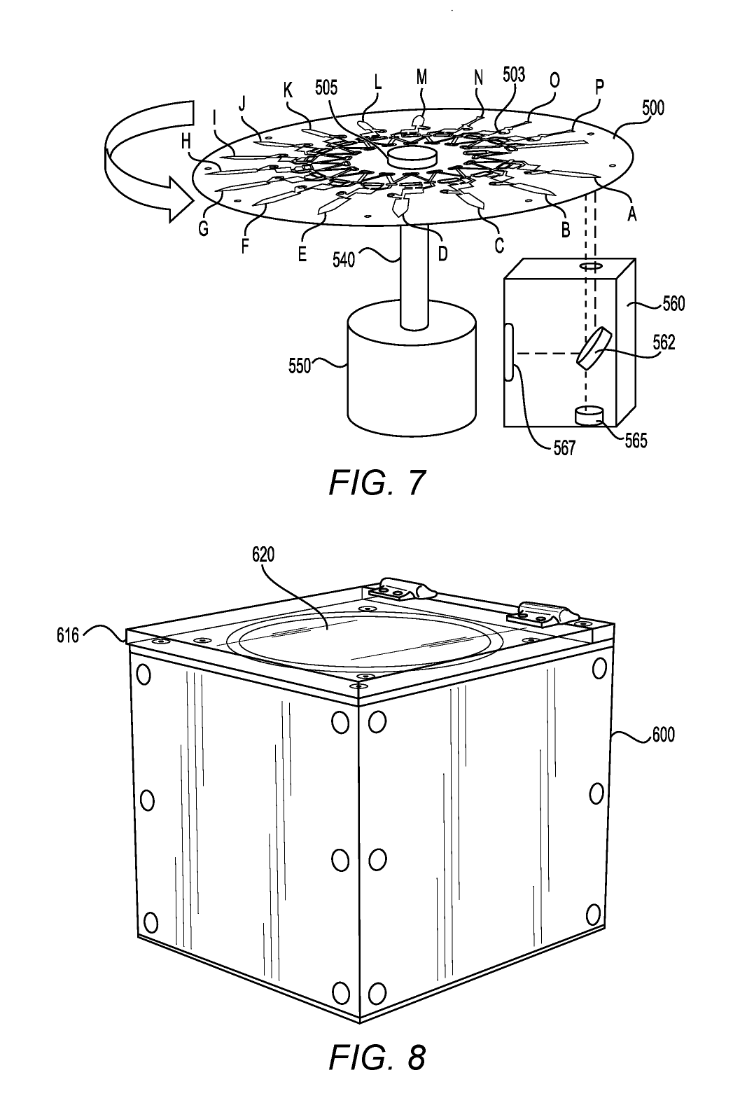

Devices according to the present disclosure may be configured to receive a microfluidic disc for performing the assays. An exemplary device is shown in FIG. 7, comprising a detection component for detecting a target (e.g., biomarker) in the various assays discussed above. As shown in FIG. 7, the device may comprise a microfluidic disc 500, a power source such as a motor 550, and a detection component 560. The disc 500 may be operably coupled to the motor 550 via a shaft 540, such that the motor 550 may power rotation of the disc 500 via the shaft 540. The motor may control rotation of the disc 500 counterclockwise (in the direction of the arrow shown in FIG. 5) and/or clockwise at a predetermined speed or series of predetermined speeds.

In some aspects, the device may be configured to heat certain chambers of the disc at a predetermined temperature or temperature gradient, such as during an amplification reaction or other type of reaction. For example, the device may include one or more heating elements in close proximity of the disc 500, e.g., above and/or below the disc 500. The position of the heating elements may correspond to the location(s) of the chamber(s) of the disc 500 to be heated, such that the heating is localized to the desired chamber(s). In some examples, the chambers may be designed such that only some of the chambers (e.g., having the same radial distance) will be heated by the heating elements, whereas other chambers will not be heated. In some aspects of the present disclosure, portions of the microfluidic disc may comprise an insulating material or heat transfer material to facilitate localized heating of chambers.

The disc 500 may include any of the features of discs 100, 140, 180, and/or 200 discussed above, including, e.g., a plurality of channels 503 and a central aperture 505. Each channel 503 may include the appropriate chambers and other features designed for the particular assay being performed. For example, the channels 503 may include respective chambers at the outermost end of the channels 503 (e.g., detection chambers or array chambers), labeled sequentially A-P in FIG. 7, which may contain the labeled targets produced by the assay to be detected.

In some examples, the detection component 560 may be configured to detect the presence of targets by measuring signals from detection molecules bound to the targets in respective chambers A-P at or proximate the edge of the disc 500. For example, the detection component 560 may detect absorbance, fluorescence, chemiluminescence, or electrochemiluminscence, or any other type of signals from a detectable label bound to the targets within the channels 503 of the disc 500. The amount of a target in each chamber A-P (and thus the concentration of the target in the original sample) may be determined based on the level of signal detected, location and/or configuration information for each chamber A-P, and/or rotation characteristics of the disc 500. For example, each chamber A-P may include reagents specific to a different type of target, such that the position of each chamber relative to the others may be used to identify the target being detected. If the collection of signal begins when the detection component 560 is aligned with chamber A, as the disc 500 rotates, the amount of signal emitted from chamber A may be distinguished from the amount of signal emitted from chambers B—P based on the speed of rotation and the location of chamber A. Thus, for each full rotation of the disc 500, the detection component 560 may collect signal for each of chambers A-P.

When microarrays are used as substrates for binding to targets, the predetermined configuration of the microarray (e.g., the number and the position of features corresponding to each target) also may be used to associate the signal measured for the microarray with the identity of the target generating the signal. When chambers A-P contain different targets (e.g., due to the use of different capture molecules and/or different detection molecules to bind to the targets, as discussed above), the concentrations of multiple targets present in the sample may be determined simultaneously or substantially simultaneously.

In some aspects, the detection component 560 may be an optical detector including a light source 565 for generating light, a detector 567, and optics 562 (e.g., mirrors and/or lenses) directing light from the light source 565 to the disc 500 and redirecting light emitted from the disc 500 to the detector 567. In at least one embodiment the detection component comprises light excitation at various wavelengths in the visible region and also outside the visible region, including, but not limited to a laser excitation, or a light-emitting diode (LED) excitation and a complementary metal-oxide semiconductor (CMOS) sensor for detection of specific wavelengths, with the use of one or more appropriate filters and/or dichroic beam-splitters.

The detection component 560 may further include a reader for analyzing data from the detector 567 and a screen for displaying output from the reader. The reader may be optical. In some embodiments, the detection component 560 may include an imaging system, e.g., comprising a charge coupled device (CCD) camera. Output from the imaging system may be displayed on a computer screen or other user interface or viewing apparatus, including, but not limited to, e.g., a liquid crystal display (LCD) device. In some aspects, output from the imaging system may be transferred to a remote user interface such as a tablet computer or other computer controlled device such as a laptop or smartphone. The data may be transferred via wire or wireless communication, including, but not limited to, Bluetooth, and/or may be stored or archived on remote servers, e.g., in the Internet cloud.

FIG. 8 shows an exemplary housing 600 of a device according to some aspects of the present disclosure. For example, the housing may contain the device of FIG. 7. In some aspects, the housing 600 may include a cover (e.g., movable via hinges as shown or other suitable mechanism) and a door 620 that may be opened and closed for inserting and removing a microfluidic disc, e.g., any of discs 100, 200, or 500.

It is intended that the specification and examples be considered as exemplary only, with a true scope and spirit of the present disclosure being indicated by the following claims.

What is claimed is:

1. A device comprising:
a disc including a plurality of microfluidic channels extending in a radial direction of the disc, each microfluidic channel comprising a plurality of capture molecules specific to at least one target chosen from an oligonucleotide, a protein, or a small molecule, wherein each capture molecule comprises an oligonucleotide and is attached to a substrate.

2. The device of claim 1, wherein the plurality of capture molecules includes at least one aptamer.

3. The device of claim 1, wherein each oligonucleotide of the plurality of capture molecules comprises a sequence at least partially complementary or fully complementary to a sequence of the at least one target.

4. The device of claim 1, wherein the plurality of capture molecules includes at least one chimeric molecule comprising an oligonucleotide.

5. The device of claim 4, wherein each oligonucleotide of the plurality of capture molecules has a length ranging from 20 nucleotides to 5,000 nucleotides.

6. The device of claim 1, wherein the plurality of capture molecules comprises natural nucleotides, synthetic nucleotides, or a combination thereof.

7. The device of claim 1, wherein the plurality of capture molecules comprises DNA or RNA.

8. The device of claim 1, wherein the substrate comprises a microarray or a plurality of microbeads.

9. The device of claim 1, wherein the substrate comprises a microarray, and the plurality of capture molecules includes a first plurality of capture molecules specific to a first target attached to a first area of the microarray and a second plurality of capture molecules specific to a second target attached to a second area of the microarray.

10. The device of claim 8, wherein the first area includes two or more discrete features on the microarray defined by a grouping of the first plurality of capture molecules.

11. The device of claim 8, wherein the microarray includes capture molecules specific to at least three different targets.

12. The device of claim 11, wherein the substrate comprises a plurality of microbeads having an average diameter ranging from 100 nm to 10 μm.

13. The device of claim 1, wherein the at least one target is a biomarker, such that the plurality of capture molecules includes capture molecules specific to biomarkers indicative of a disease.

14. The device of claim 1, wherein the plurality of capture molecules includes capture molecules specific to biomarkers indicative of cancer, a cardiac disease, a respiratory disease, a neurological disease, an infectious disease, or antibiotic resistant genes, wherein the plurality of capture molecules includes capture molecules specific to pathogens associated with an infectious disease.

16. A method for detecting at least one target in a fluid sample using the device, comprising:
introducing the fluid sample into at least one microfluidic channel of the disc;
rotating the disc, such that the fluid sample flows radially outward through the at least one microfluidic channel to combine with at least one capture molecule of the plurality of capture molecules, wherein the fluid sample comprises blood or is obtained from blood; and
detecting a signal from the disc indicative of a presence of at least one target in the sample.

17. The method of claim 16, wherein the at least one target comprises an oligonucleotide, a protein, or a small molecule.

18. The method of claim 16, wherein the plurality of capture molecules includes at least one aptamer that binds to the at least one target.

19. The method of claim 16, wherein the plurality of capture molecules includes at least one oligonucleotide that hybridizes to the at least one target.

20. The method of claim 16, wherein amplifying the at least one target includes performing a polymerase chain reaction or an isothermal amplification process.
